(19) United States
(12) Patent Application Publication
(10) Pub. No.: US 2022/0184290 A1
(43) Pub. Date: Jun. 16, 2022

(54) SYSTEMS AND METHODS FOR TREATING PATIENTS WITH (OR AT RISK FOR) ABNORMAL BLOOD CLOTS, PARTICULARLY COVID-19 PATIENTS

(71) Applicant: Regina E. Herzlinger, Belmont, MA (US)

(72) Inventor: Regina E. Herzlinger, Belmont, MA (US)

(21) Appl. No.: 17/549,800

(22) Filed: Dec. 13, 2021

Related U.S. Application Data
(60) Provisional application No. 63/125,301, filed on Dec. 14, 2020.

(51) Int. Cl. A61M 1/34; A61M 1/36

(52) U.S. Cl. CPC A61M 1/34; A61M 2205/273; A61M 1/3673

(57) ABSTRACT

The present disclosure provides, among other things, methods and systems for treating or removing blood clots in a biological fluid. In some embodiments, the present disclosure provides dialysis-like systems for capturing blood clots. The present disclosure encompasses the recognition that such methods and systems are useful for patients at risk for pulmonary embolism (e.g., patients with deep vein thrombosis and/or COVID-19 patients).

### CROSS REFERENCE TO RELATED APPLICATIONS

The present application claims priority to U.S. Provisional Patent Application No. 63/125,301, filed on Dec. 14, 2020, the entire disclosure of which is incorporated herein by reference.

### BACKGROUND

Abnormal blood clots are associated with a number of life-threatening health conditions, including heart attack, stroke, and pulmonary embolism. Many patients hospitalized with COVID-19 (the disease caused by the virus SARS-CoV-2) develop abnormal blood clotting, including pulmonary embolism. COVID-19 patients that exhibit symptoms of pulmonary embolism are more likely to require ICU treatment. Pulmonary embolism can restrict blood flow to the lungs, decreasing oxygen levels in the blood and in some cases, can be fatal. Current methods to treat blot clots may be of questionable efficacy and may be potential harmful to certain patients, such as COVID-19 patients. Thus, there is a continuing critical need for systems and methods of treatment of blood clots, such as those associated with embolism (e.g., pulmonary embolism), particularly in COVID-19 patients.

### SUMRIARY

The present disclosure provides systems and methods for treating patients with or at risk for developing abnormal blood clots (e.g., associated with embolism, e.g., pulmonary embolism). The present disclosure recognizes limitations of prior systems, such as an IVC (inferior vena cava) filter, which can migrate, fracture, or cause extensive scar formation. (See e.g., Williams “Mayo Clinic Minute: Ins and outs of blood clot filters” Jul. 18, 2019, https://newsnetwork.mayoclinic.org/discussion/mayo-clinic-minute-ins-and-outs-of-blood-clot-filters/). The present disclosure provides, among other things, methods and systems for treating or removing blood clots in a biological fluid (e.g., blood). In some embodiments, the present disclosure provides dialysis-like systems for capturing blood clots. In such dialysis-like systems, blood is drawn from a patient via a pump. The blood flows through a filter ex vivo and then the filtered blood is returned to the patient. In some embodiments, provided filters can capture blood clots (e.g., blood clots above a threshold size) from the blood of a patient. The present disclosure encompasses the recognition that such methods and systems are useful for patients at risk for pulmonary embolism (e.g., patients with deep vein thrombosis and/or COVID-19 patients). For example, in some embodiments, provided systems and methods can capture one or more blood clots that have broken off from a deep vein thrombosis (DVT) and are migrating in a patient's circulatory system. In some embodiments, a patient with DVT exhibits an elevated level of D-dimer (e.g., >1,000 μg/L), which can be detected and used to signal filtration is needed and/or a blood clot is about to be, or may have been, filtered by the system.

In some aspects, the present disclosure recognizes that such methods and systems may be particularly useful for treating COVID-19 patients with abnormal blood clots. In some embodiments, a COVID-19 patient has or is suspected to have DVT. In some embodiments, provided systems and methods can capture a blood clot that has broken off from a DVT and is migrating in a COVID-19 patient's circulatory system. In some embodiments, a COVID-19 patient exhibits an elevated level of D-dimer (e.g., >1,000 μg/L).

In one aspect, the invention is directed to a system for treating a patient having or at risk of having abnormal blood clotting (e.g., deep vein thrombosis, unexplained hypotension, unexplained tachycardia, unexplained worsening respiratory status, traditional risk factors for thrombosis (e.g., history of thrombosis, cancer, hormonal therapy) and/or wherein the patient has COVID-19), the system comprising: a continuous-flow pump comprising an intake port and an outtake port (e.g., two ports that enable continuous blood flow such that it is unnecessary to create a shunt by linking an artery and a vein as with dialysis); and a disposable set comprising tubing and a filter for capturing blood clots.

The present disclosure provides that any suitable vein (as determined by one of skill in the art) can serve as an input source for a system of the present disclosure to draw blood from the patient, and any suitable artery as a output to return blood to the patient. In some embodiments, blood is drawn from the inferior vena cava to input into the system. In some embodiments, blood is drawn from an iliac vein, such as a common iliac vein, to input into the system. In some embodiments, blood is drawn from a femoral vein to input into the system,

In some embodiments, blood is advantageously input into the system through a connection to a vein that is past a DVT in a return flow of blood to the heart, to permit capture of the thrombus by the filter.

In some embodiments, the system also comprises a sensor for detecting D-dimer (e.g., and/or other species) in blood as a marker of clots (e.g., a DVT marker) and/or toxic blood substances (e.g., an electrochemical impedance detector for monitoring D-dimer association with a biosensor, e.g., a single-chain antibody (ScAb) immobilized on a transducer surface with a receptor layer) (e.g., wherein an elevated D-dimer level is suggestive of the presence of one or more clots, e.g., deep vein thrombosis clots).

In some embodiments, the system also comprises a mechanism for signaling when the filter is to be changed (e.g., an alarm). In some embodiments, an alarm will sound when a clot is captured. In some embodiments, the mechanism for signaling when the filter is to be changed initiates an automated process for changing the filter.

In some embodiments, the system further comprises an anti-thrombotic agents to reduce and/or eliminate build-up on pump surfaces that come into contact with blood (e.g., tubing interior). In certain embodiments, the system further comprises one or more anti-thrombotic agents to reduce and/or eliminate build-up on pump surfaces that come into contact with blood. In certain embodiments, the system comprises two or more anti-thrombotic agents to reduce and/or eliminate build-up on pump surfaces that come into contact with blood. In certain embodiments, the anti-thrombotic agent(s) comprises one or more of low-molecular weight (e.g., 6 kDa) propylene glycol alginate sodium sulfate (PSS), prostacyclin, or heparin (e.g., including low molecular weight heparin).

In certain embodiments, the pump is capable of providing a maximum continuous blood flow of at least 200 mL/min (e.g., at least 300 mL/min, at least 400 mL/min, at least 500 mL/min, at least 600 mL/min, at least 700 mL/min, at least 800 mL/min, at least 900 mL/min, at least 1000 mL/min, at least 1100 mL/min, at least 1200 mL/min, at least 1300 mL/min, at least 1400 mL/min or at least 1500 ml/min). In certain embodiments, the pump is capable of providing a continuous blood flow at a rate within a range of between 300 mL/min and 4 L/min.

In certain embodiments, the filter comprises one or more members selected from the group consisting of cellulose, synthetic polymer, and graphene (e.g., graphene for capturing very small, micrometer-scale clots) (e.g., wherein the filter has a membrane structure and/or hollow-fiber structure). In some embodiments, the filter has a structure capable of capturing blood clots of size within a range between 100 micrometers and 10 mm.

In another aspect, the invention is directed to a method of treating a patient having or at risk of having a blood clotting disorder (e.g., wherein the patient has COVID-19), the method comprising: filtering blood of the patient (e.g., using a system described herein) in order to capture blood clots (e.g., clots that present a risk of pulmonary embolism) (e.g., continuously filtering over the course of a treatment).

In some embodiments, a patient to be treated with a method and/or system of the present disclosure has a DVT and/or COVID-19. In some embodiments, the patient further comprises another cardiopulmonary disease such as Cardiovascular Disease (CVD) and/or Chronic Obstructive Pulmonary Disorder (COPD).

In certain embodiments, the filter has a structure capable of capturing blood clots of size within a range of about 100 micrometers to about 5000 micrometers. In certain embodiments, the filter has a structure capable of capturing blood clots of size (i) 100 micrometers or larger, or (ii) 200 micrometers or larger, or (iii) 300 micrometers or larger, or (iv) 400 micrometers or larger, or (v) 500 micrometers or larger, or (vi) 600 micrometers or larger, or (vii) 800 micrometers or larger, or (viii) 1000 micrometers or larger. In certain embodiments, smaller clots (e.g., less than 200 micrometers), sometimes referred to as “micro-clots”, can be captured by the filter, for example, where the filter selectively adsorbs or absorbs the micro-clot while allowing other similar-sized components of the blood to pass through, to be returned to the body. In certain embodiments, a COVID-19 patient may be at risk of developing such micro-clots. In some cases, multiple DVT clots and/or other clots may accumulate to form a dangerous pulmonary embolism; thus, in certain embodiments, the filter system can prevent or delay formation of a pulmonary embolism by collecting clots from the bloodstream before they have a chance to accumulate and/or lodge in a location to form a pulmonary embolism.

In some embodiments, the method comprises obtaining, determining, or monitoring one or more procoagulation factors in the blood of the patient. Procoagulation factors include D-dimer, Partial Thromboplastin Time (PTT), prothrombin time (PT), and fibrinogen.

In certain embodiments, the method comprises monitoring for the presence of D-dimer (e.g., and/or other species) in the blood being pumped as a marker of clots in the blood and/or procoagulation disorders. In certain embodiments, an alarm is sounded and/or a visual alert is provided to alert an attendant/operator/medical staff that a clot has been detected and/or captured. In certain embodiments, the alarm and/or visual alert identifies a risk that a clot may be present in the blood flow, e.g., a clot that is about to be captured by the filter.

In certain embodiments, the method further comprises administering an anticoagulant to the patient. In certain embodiments, the anticoagulant is administered through a one-way pump rather than a continuous-flow pump. In certain embodiments, the anticoagulant is not administered via a pump but is otherwise administered (e.g., intravenously). In certain embodiments, the anticoagulant comprises a member selected from the group consisting of heparin (including low molecular weight heparin), warfarin (Coumadin), rivaroxaban (Xarelto), dabigatran (Pradaxa), apixaban (Eliquis), and edoxaban (Lixiana).

In certain embodiments, the method comprises replacing the filter (e.g., replacing the disposable set comprising the filter) upon capture of one or more clots from the blood.

Throughout the description, where systems or compositions are described as having, including, or comprising specific components, or where methods are described as having, including, or comprising specific steps, it is contemplated that, additionally, there are systems or compositions of the present invention that consist essentially of, or consist of, the recited components, and that there are methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The following description is for illustration and exemplification of the disclosure only, and is not intended to limit the invention to the specific embodiments described.

The mention herein of any publication, for example, in the Background section, is not an admission that the publication serves as prior art with respect to any of the claims presented herein. The Background section is presented for purposes of clarity and is not meant as a description of prior art with respect to any claim.

### BRIEF DESCRIPTION OF THE DRAWING

FIG. 1 depicts an exemplary schematic of a continuous flow system for use in the context of the present disclosure, according to an illustrative embodiment.

### DETAILED DESCRIPTION

Abnormal blood clots are associated with a number of life-threatening health conditions, including heart attack, stroke, and pulmonary embolism (“PE”). The present disclosure provides methods and systems for attenuating and/or mitigating the risk of such life-threatening conditions in patients with deep vein thrombosis (“DVT”) or other blood clot disorder. Patients with DVT have an attendant risk of embolism (e.g., PE). Without wishing to be limited by theory, it is recognized that in some cases a portion of the DVT can break off and migrate through the circulatory system. Such migrating clots may become lodged in an organ (e.g., lung in PE), causing a restriction in blood flow, which can potentially be fatal. The present disclosure provides methods and systems for capturing such migrating clots (e.g., those that have broken off of a DVT).

Recently, it was recognized that COVID-19 (the disease caused by the virus SARS-CoV-2) is associated with a risk of developing DVT and/or PE. For example, it was reported that approximately a quarter of patients hospitalized with severe COVID-19 had signs of PE. Grillet et al. (Apr. 23, 2020) Acute Pulmonary Embolism Associated with COVID-19 Pneumonia Detected by Pulmonary CT Angiography, Radiology, https://doi.org/10.1148/radiol.2020201544.

COVID-19 patients that exhibit symptoms of PE have worse prognoses and are more likely to require ICU treatment. For example, patients with PE have been reported to be more frequently in the critical care unit than those without pulmonary embolus (17 (74%) vs 22 (29%) patients, p<0.001) and also required mechanical ventilation more often (15 (65%) versus 19 (25%) patients, p<0.001). Moreover, this increased requirement for mechanical ventilation has been associated with acute PE. Id.

Current methods to treat blood clots are of questionable efficacy and may be potential harmful to certain patients (e.g., COVID-19 patients). For example, surgical insertion of an IVC (inferior vena cava) filter to help prevent blood clots from entering the lungs is invasive and associated with a number of device risks such as breakage, migration, and scarring. In some instances, an IVC can break inside a patient and perforate blood vessels. Moreover, IVCs are of questionable efficacy. Anticoagulants can be administered in an attempt to prevent blood clots from forming, but these drugs are associated with risk of excessive bleeding (i.e., hemorrhage), may not be suitable for treatment of all patients, and in COVID-19 patients it is still unclear if these are treatments are effective. High bleeding complications have also been reported with full-dose systemic thrombolysis, which may not be suitable for COVID-19 patients.

The present disclosure provides methods and systems useful for treating or removing blood clots in a biological fluid (e.g., blood). In some embodiments, provided are novel dialysis-like systems for capturing blood clots, and methods of using the same to treat patients (e.g., patients with DVT and/or COVID-19 patients).

### Patients Having or at Risk for Abnormal Blood Clots

The present disclosure provides methods and systems useful for capturing blood clots in a biological fluid (e.g., blood) of a patients in need thereof. In some embodiments, such patients have or are at risk for embolism (e.g., pulmonary embolism). Pulmonary embolism (“PE”) occurs when a clump of material, most often a blood clot, builds up and/or gets wedged into an artery of the lungs. These blood clots most commonly originate from a condition known as deep vein thrombosis (DVT). Exemplary description of pulmonary embolism can be found on the Mayo Clinic website at: https://www.mayoclinic.org/diseases-conditions/pulmonary-embolism/symptoms-causes/syc-20354647_.

In some embodiments, patients that have or are at risk for PE include patients with DVT and/or COVID-19 patients.

In some embodiments, a patient to be treated with a method and/or system of the present disclosure has DVT and/or COVID-19. In some embodiments, the patient further has another cardiopulmonary disease such as Cardiovascular Disease (CVD) and/or Chronic Obstructive Pulmonary Disorder (COPD). Use of the systems and/or methods described herein may be part of an active treatment for a confirmed blood clot condition, or may be employed prospectively at a particular stage of disease, e.g., the systems and/or methods may be used to treat a COVID-19 patient when the risk of DVT and/or PE is substantial, even if abnormal blood clots have not yet been confirmed.

Markers for Abnormal Blood Clots

The present disclosure recognizes that levels of certain procoagulation factors in the blood of the patient can serve as a marker for a patient that has or is at risk for abnormal blood clots, e.g., embolism, e.g., PE. In some embodiments, a level of one or more procoagulation factors in the blood of the patient is obtained, determined, and/or monitored. Procoagulation factors include D-dimer, Partial Thromboplastin Time (PTT), prothrombin time (PT), and fibrinogen.

In some certain embodiments, a level of D-dimer is in the blood of the patient is obtained, determined, and/or monitored. D-dimer is a small protein fragment (a fibrin degradation product) that present in the blood upon degradation of a blood clot by fibrinolysis. D-dimer is normally undetectable or at a very low level unless the body is forming and breaking down blood clots. A normal level of D-dimer provides reasonable confidence that PE and/or DVT are not present. In some embodiments, an elevated level of D-dimer in the blood of a patient serves as a marker for abnormal clots (e.g. suggestive of DVT). However, an elevated D-dimer level in and of itself is not conclusive of a diagnosis of PE and/or DVT. For example, in a COVID-19 patient and elevated D-dimer level may result from other causes (e.g., secondary infection, myocardial infarction, renal failure, or coagulopathy). Further description of elevated D-dimer level in a COVID-19 patient can be found at, e.g., https://www.hematology.org/covid-19/covid-19-and-pulmonary-embolism

In some embodiments, the patient has a level of D-dimer that is at least 500 μg/L, at least 600 μg/L, at least 700 μg/L, at least 800 μg/L, at least 900 μg/L, at least 1,000 μg/L, at least 2,000 μg/L, at least 3,000 μg/L, at least 4,000 μg/L, or at least 5,000 μg/L. In some embodiments, a patient has a level of D-dimer that is within a range of 500 μg/L to 250,000 μg/L, 1,000 μg/L to 250,000 μg/L, 2,000 μg/L to 250,000 μg/L, 5,000 μg/L to 250,000 μg/L, 1,000 μg/L to 2,000 μg/L, 1,000 μg/L to 4,000 μg/L, 2,000 μg/L to 4,000 μg/L, 1,000 μg/L to 5,000 μg/L, 2,000 μg/L to 10,000 μg/L, 2,000 μg/L to 5,000 μg/L, or 4,000 μg/L to 10,000 μg/L. In certain embodiments, the patient has a high level of D-dimer that is characterized as greater than 5,000 μg/L (e.g., within a range of 5,000 μg/L to 250,000 μg/L). Schutte et al., Neth. J. Med. 2016 December; 74(10):443-448.

In some embodiments, a level of D-dimer is measured using: (i) quantitative fluorescence immunoassay, (ii) quantitative microparticle enzyme immunoassay (MEIA) chemiluminescence, (iii) quantitative, latex-enhanced immunoturbidimetric immunoassay, (iv) quantitative solid-phase immunochromatography, (v) enzyme-linked immunosorbent assay (ELISA), and/or (vi) quantitative latex agglutination. Riley, et al., Laboratory Medicine, Volume 47, Issue 2, 1 May 2016, Pages 90-102.

Patients with Deep Vein Thrombosis

The present disclosure encompasses the recognition that provided methods and systems are useful for patients with or at risk for deep vein thrombosis (“DVT”). DVT occurs when a blood clot (also referred to as a thrombus) forms in one or more deep veins of a patient (e.g., in the legs). DVT can present with leg pain or swelling in a patient, but also can occur with no symptoms or difficult-to-detect symptoms. Accordingly, diagnosis of DVT usually includes testing such as, e.g., detection of a biomarker for DVT (e.g., D-dimer) or imaging (e.g., ultrasound, venography, CT scan and/or MRI scan).

DVT is associated with a risk of PE. For example, part of a DVT can break away and this piece of thrombus (i.e. blood clot) can move through the circulatory system. If this circulating piece of thrombus (i.e. blood clot) reaches a blood vessel that is too small to let it pass, it can become stuck and block blood flow. PE occurs when a thromboembolus (blood clot) is carried to the lung and blocks a pulmonary artery. When a PE blocks a pulmonary artery, it causes respiratory distress that is proportional to the amount of blockage, e.g., to the size of the embolus. Large emboli that block both pulmonary arteries can cause immediate death.

In some embodiments, the DVT patient has or is at risk for developing a thromboembolus (i.e., a blood clot breaking off from the DVT). In some embodiments, a thromboembolus is of a size that is within a range bounded by a lower limit and an upper limit, the upper limit being larger than the lower limit. In some embodiments, the lower limit may be 100 μm, 200 μm, 300 μm, 400 μm, 500 μm, 600 μm, 700 μm, 800 μm, 900 μm, or 1 mm. In some embodiments, the upper limit may be 200 μm, 300 μm, 400 μm, 500 μm, 600 μm, 700 μm, 800 μm, 900 μm, 1 mm, 2 mm, 3 mm, 4 mm, 5 mm, 6 mm, 7 mm, 8 mm, 9 mm, or 10 mm.

In some embodiments, a patient to be treated with a system or method of the present disclosure has been diagnosed as having a DVT or likely to have a DVT. In some embodiments, the patient is diagnosed with a DVT by imaging, such as ultrasound, venography, CT scan and/or MRI scan. In some embodiments, the patient is diagnosed with a DVT by the presence of D-dimer (e.g., a level of D-dimer above a threshold level).

In some embodiments, the DVT patient has a level of D-dimer that is at least 500 μg/L, at least 1,000 μg/L, at least 2,000 μg/L, at least 3,000 μg/L, at least 4,000 μg/L, or at least 5,000 μg/L. In some embodiments, a patient has a level of D-dimer that is within a range of 500 μg/L to 250,000 μg/L, 1,000 μg/L to 250,000 μg/L, 2,000 μg/L to 250,000 μg/L, 5,000 μg/L to 250,000 μg/L, 1,000 μg/L to 2,000 μg/L, 1,000 μg/L to 4,000 μg/L, 2,000 μg/L to 4,000 μg/L, 1,000 μg/L to 5,000 μg/L, 2,000 μg/L to 10,000 μg/L, 2,000 μg/L to 5,000 μg/L, or 4,000 μg/L to 10,000 μg/L. In certain embodiments, the patient has a high level of D-dimer that is characterized as greater than 5,000 μg/L (e.g., within a range of 5,000 μg/L to 250,000 μg/L). Schutte et al., Neth. J. Med. 2016 December; 74(10):443-448.

In some embodiments, a level of D-dimer is measured in a DVT patient using: (i) quantitative fluorescence immunoassay, (ii) quantitative microparticle enzyme immunoassay (MEIA) chemiluminescence, (iii) quantitative, latex-enhanced immunoturbidimetric immunoassay, (iv) quantitative solid-phase immunochromatography, (v) enzyme-linked immunosorbent assay (ELISA), and/or (vi) quantitative latex agglutination. Riley, et al., Laboratory Medicine, Volume 47, Issue 2, 1 May 2016, Pages 90-102.

Coronavirus Patients

In some aspects, the present disclosure recognizes that such methods and systems may be particularly useful for treating patients with an illness caused by a coronavirus, such as COVID-19 (the disease caused by the virus severe acute respiratory syndrome coronavirus-2, also known as SARS-CoV-2), who exhibit abnormal blood clots (e.g., DVT and/or PE). As noted above, certain COVID-19 patients exhibit symptoms of DVT and/or PE, and these patients have worse prognoses and are more likely to require ICU treatment.

In some embodiments, a coronavirus patient (e.g., COVID-19 patient) to be treated with a method and/or system described herein has a level of D-dimer that is at least 500 μg/L, at least 1,000 μg/L, at least 2,000 μg/L, at least 3,000 μg/L, at least 4,000 μg/L, or at least 5,000 μg/L. In certain embodiments, a COVID-19 patient has a level of D-dimer that is at least 1,000 μg/L.

In some embodiments, a coronavirus patient (e.g., COVID-19 patient) to be treated with a method and/or system described herein has a level of D-dimer that is within a range of 500 μg/L to 250,000 μg/L, 1,000 μg/L to 250,000 μg/L, 2,000 μg/L to 250,000 μg/L, 5,000 μg/L to 250,000 μg/L, 1,000 μg/L to 2,000 μg/L, 1,000 μg/L to 4,000 μg/L, 2,000 μg/L to 4,000 μg/L, 1,000 μg/L to 5,000 μg/L, 2,000 μg/L to 10,000 μg/L, 2,000 μg/L to 5,000 μg/L, or 4,000 μg/L to 10,000 μg/L. In certain embodiments, the patient has a high level of D-dimer that is characterized as greater than 5,000 μg/L (e.g., within a range of 5,000 μg/L to 250,000 μg/L). Schutte et al., Neth. J. Med. 2016 December; 74(10):443-448. Oudkerk et al. Radiology, Apr. 23, 2020, https://doi.org/10.1148/radiol.2020201629.

In some embodiments, a coronavirus patient (e.g., COVID-19 patient) to be treated with a method and/or system described herein exhibits hypoxemia.

In some embodiments, the coronavirus patient (e.g., COVID-19 patient) exhibits reduced pulmonary compliance (i.e., impaired ability of the lungs to stretch and expand). Total compliance of both lungs in a typical healthy adult is about 200 ml/cmH₂O. In some embodiments, the coronavirus patient (e.g., COVID-19 patient) exhibits a pulmonary compliance value that is less than 100 ml/cmH₂O, less than 90 ml/cmH₂O, less than 80 ml/cmH₂O, less than 70 ml/cmH₂O, less than 60 ml/cmH₂O, less than 50 ml/cmH₂O, or less than 40 ml/cmH₂O.

In some embodiments, a subject for treatment with a method and/or system of the present disclosure has coronavirus (e.g., COVID-19 patient) and has or is suspected to have DVT. In some embodiments, the coronavirus patients (e.g., COVID-19 patient) has or is at risk for thromboemboli and/or PE.

### Systems

In another aspect, the invention is directed to a system for treating a biological fluid of a subject having abnormal blood clotting (e.g., DVT, PE, and/or COVID-19). In some embodiments, the provided system is a dialysis-like system useful for removing blood clots in a biological fluid (e.g., blood).

In some embodiments, a system comprises: a continuous-flow pump comprising an intake port and an outtake port (e.g., two ports such that it is unnecessary to create a shunt by linking an artery and a vein as with dialysis); a disposable set comprising tubing and a filter for capturing blood clots.

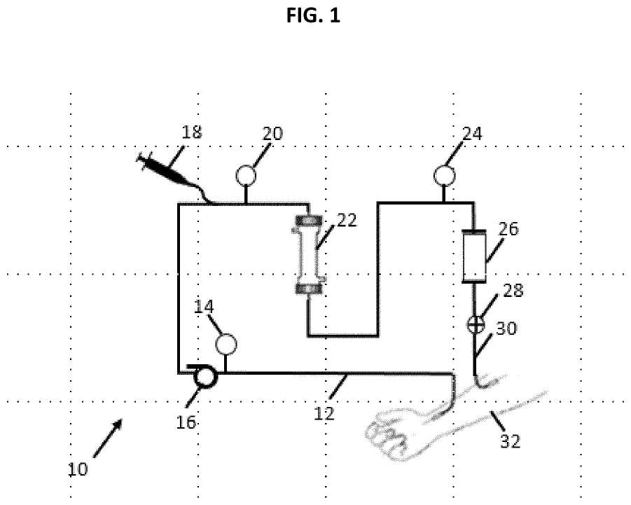

FIG. 1 depicts an illustrative arrangement of system components. In this illustrative embodiment, blood is drawn from the subject 32 by a pump 16 into a tubing line 12. In some embodiments, the pump 16 provides for reduction or elimination of microbubbles that are present in the blood prior to being contacted with the filter 22. Optionally, heparin may be administered in the blood flow line at the heparin pump 18 to prevent clotting. Other anti-coagulants may alternatively or additional be used, such as CPDA-1, CPD, ACD, and/or Hirudin. The blood passes through a blood clot-capturing filter 18, described further herein. Treated blood then exits the filter and passes through an air trap/detector 26 before it is returned to the subject. The system may also include pressure monitors 14, 20, 24 (arterial pressure, inflow pressure, and venous pressure). In certain embodiments, components that come into contact with the biological fluid are biocompatible and/or sterilizable and/or replaceable. In certain embodiments, the pump 16 receives electric power by an Alternating Current (AC) wall outlet. In certain embodiments, the pump 16 is operated from battery power.

In certain embodiments, the pump 16 has a mechanical and/or electronic operation mechanism. In certain embodiments, the pump 16 has a pumping mechanism design classified as one or more of the following: rotary, roller, peristaltic, centrifugal, elastomeric, syringe, diaphragm, or piston. In certain embodiments, the pump provides sufficiently high flow to effectively and/or efficiently filter out clots. In certain embodiments, the pump 16 comprises a pump (and/or one or more other components) such as those used in current rapid infuser systems. Examples of rapid infuser systems include the Hotline HL-1200A Rapid Infuser Infusion Pump (capable of infusion rates from 30 mL/min to 1100 mL/min, with maximum rate of 1400 mL/min) (Smiths Group Plc, London, UK); the Belmont® Rapid Infuser RI-2 (capable of infusion rates from 2.5 mL/min to 1000 mL/min), the FMS2000, the buddy™ and the buddy lite™ portable IV & infusion pump (Belmont Medical Technologies, Billerica, Mass.); LifeFlow Rapid Fluid Infuser, and LifeFlow Plus Rapid Fluid and Blood Infuser (capable of 500 mL of fluid in less than 2 min, 20 G IV catheter, or 274 mL/min via 18ga catheter) (410 Medical, Durham, N.C.); Thermacor 1200 (capable of infusion rates from 10 mL/hour to 1200 mL/min) (Smisson-Cartledge Biomedical, Macon, Ga.); The Warrior lite, Warrior, Warrior EXTREME, Warrior Hybrid, and Warrior AC (QinFlow Ltd. of Rosh Ha'ayin Israel); enFlow® IV fluid and blood warming system (CareFusion, Vernon Hills, Ill.); Medi-Temp by Stryker (Kalamazoo, Mich.); Ranger by 3M (St. Paul, Minn.); Level 1 h-1200 Fast Flow Fluid Warmer (Smiths Medical, Dublin, Ohio); and Thermal Angel® blood and IV fluid infusion warmer (Estill Medical Technologies, Inc., Arlington, Tex.). Devices with proprietary tubing sets include the enFlow with a 4-mL priming volume and a flow rate up to 200 mL/minute; the Medi-Temp with a flow rate up to 500 mL/minute; and the Ranger by 3M (St. Paul, Minn.) with a flow rate up to 500 mL/minute. The portable Belmont® buddy™ system is designed for flow rates up to 100 mL/min for crystalloids at 20° C. and up to 50 mL/min for packed red cells at 10° C. The portable, battery powered buddy lite™ system is designed for maximum flow rates of 50-80 mL/min, depending on the input temperature. Pressurized devices for massive transfusion of blood include the Belmont Rapid Infuser RI-2 which can deliver a flow rate of more than 750 mL/minute (e.g., up to 1500 mL/minute); the Level 1 h-1200 Fast Flow Fluid Warmer which can infuse fluids at flows of up to 600 mL/min. Many of the above devices (including the portable devices) include a flow control system and/or other flow and/or metering control devices, such as pressure-regulating valves (PRVs) and/or pressure-responsive valves, to control the specific flow rate of a liquid delivered to the patient and/or to ensure the flow stays below a predetermined maximum flow rate and/or above a predetermined minimum flow rate. Moreover, these flow control devices and/or systems may allow the operator to establish an initial lower flow rate, then increase to a safe higher flow rate.

The system of FIG. 1 depicts the system including two ports for continuous flow of blood from and to a subject through the arm, but this illustration is not to be limiting. As is generally understood, oxygenated blood from the heart travels through arteries and capillaries, which is then returned through the network of veins. The inferior vena cava brings blood from the abdomen and legs into the heart. The present disclosure provides that any suitable vein (as determined by one of skill in the art) can serve as an input source for a system of the present disclosure, and any suitable artery as a output to return blood to the patient. The present disclosure provides that any suitable vein (as determined by one of skill in the art) can serve as an input source for a system of the present disclosure, and any suitable artery as a output to return blood to the patient. In some embodiments, blood is drawn from the inferior vena cava to input into the system. In some embodiments, blood is drawn from an iliac vein, such as a common iliac vein, to input into the system. In some embodiments, blood is drawn from a femoral vein to input into the system,

In some embodiments, blood is input into the system through a connection to a vein that is past a DVT in a return flow of blood to the heart (e.g., leg, groin, chest).

In some embodiments, blood is drawn using a venous catheter.

In some embodiments, the system pumps fluid (e.g., blood) at rates within a range bounded by a lower limit and/or an upper limit, e.g., the upper limit being larger than the lower limit. In some embodiments, the lower limit may be about 100 ml/min, about 150 ml/min, about 200 ml/min, about 300 ml/min, about 400 ml/min, about 500 ml/min, about 600 ml/min, about 700 ml/min, about 800 ml/min, about 900 ml/min, about 1000 ml/min, about 1200 ml/min, about 1400 ml/min, about 1500 ml/min, about 1600 ml/min, about 1800 ml/min, or about 2000 ml/min. In some embodiments, the upper limit may be about 100 ml/min, about 150 ml/min, about 200 ml/min, about 300 ml/min, about 400 ml/min, about 500 ml/min, about 600 ml/min, about 700 ml/min, about 800 ml/min, about 900 ml/min, about 1000 ml/min, about 1200 ml/min, about 1400 ml/min, about 1500 ml/min, about 1600 ml/min, about 1800 ml/min, about 2000 ml/min, about 2500 ml/min, about 3000 ml/min, about 3500 ml/min, about 4000 ml/min, about 4500 ml/min, or about 5000 ml/min.

Variations of this system may also be implemented. For example, in certain embodiments, the biological fluid (e.g., blood) is not directly drawn from arm of the subject 32, but is drawn from another part of the body (e.g., leg, groin, chest). In some embodiments, blood is drawn through a venous catheter. In certain embodiments, blood is not drawn by a pump 16 but may travel through the filter 22 via gravity (in which case, in certain embodiments, air bubbles, e.g., microbubbles, may be removed from the biological fluid prior to its passing through the filter via an air bubble removal device other than a pump). In certain embodiments, treated blood is not immediately returned to the subject 32 but is stored, e.g., in a blood storage container. Various other biological fluids may be treated with embodiments of the system described above [e.g., blood (e.g., unmodified whole blood or modified blood), serum, plasma, cerebrospinal fluid, lymph, synovial fluid, and amniotic fluid].

In certain embodiments, the system includes a pump 16 or other device for removal of microbubbles from the biological fluid before the fluid passes through the treatment filter 22. The system may additionally have an air trap 26 and/or air detector 28 through which the treated biological fluid passes before being returned to the subject 32 (or before being stored), but, in certain embodiments, it is important that air bubbles be removed from the blood or other biological fluid prior to being introduced to the filter. In certain embodiments, the pump 16 (or other device) eliminates air bubbles present in the biological fluid before the fluid passes into the filter for treatment.

In some embodiments, the filter 22 comprises a cartridge (e.g., a replaceable cartridge). In certain embodiments, the system comprises a sensor with an alarm or other indicator that identifies when a blood clot has been captured by the filter 22. In some embodiments, the alarm may indicate when the filter has captured a threshold amount or volume of blood clots, thereby indicating to an operator when the filter is to be changed.

In certain embodiments, the filter 22 comprises a membrane (e.g., a cartridge comprising a membrane structure) (e.g., as opposed to umbrella-shaped or otherwise-shaped wire devices currently used as IVC filters). In certain embodiments, the membrane is synthetic, e.g., it is made with one or more of the following materials: polysulfone, polycarbonate, polyamide, polyethersulfone, polyacrylonitrile, and polymethylmethacrylate. In certain embodiments, the membrane is made of unmodified cellulose (e.g., cuprophan, cellulose diacetate, and/or cuprammonium rayon) and/or modified/regenerated cellulose (e.g., hemophan and/or cellulose triacetate). In certain embodiments, the membrane has a pore size and structure that satisfactorily captures the blood clots, e.g., thromboemboli that have broken off of a DVT, while accommodating the needed minimum blood flow rate for safe monitoring and capture of blood clots. In certain embodiments, the filter 22 comprises a high-flux membrane that accommodates higher rates of blood flow through the system, e.g., at least 500 mL/min, at least 600 mL/min, at least 700 mL/min, at least 800 mL/min, at least 900 mL/min, at least 1000 mL/min, at least 1100 mL/min, at least 1200 mL/min, at least 1300 mL/min, at least 1400 mL/min, at least 1500 mL/min, at least 1600 mL/min, at least 1700 mL/min, at least 1800 mL/min, at least 1900 mL/min, at least 2000 mL/min, at least 2500 ml/min, at least 3000 ml/min, at least 3500 ml/min, or at least 4000 ml/min.

In some embodiments, the system also comprises a sensor for detecting D-dimer (e.g., and/or other species) in blood as a marker of clots (e.g., a DVT marker) and/or procoagulation disorders. In some embodiments, the sensor for detecting D-dimer comprises: an electrochemical impedance detector for monitoring D-dimer association with a biosensor (e.g., a single-chain antibody (ScAb) immobilized on a transducer surface with a receptor layer) (e.g., wherein an elevated D-dimer level is indicative of the presence of clots in the blood and/or DVT.

In some embodiments, a biosensor capable of detecting D-dimer uses: (i) quantitative fluorescence immunoassay, (ii) quantitative microparticle enzyme immunoassay (MEIA) chemiluminescence, (iii) quantitative, latex-enhanced immunoturbidimetric immunoassay, (iv) quantitative solid-phase immunochromatography, (v) enzyme-linked immunosorbent assay (ELISA), and/or (vi) quantitative latex agglutination to detect D-dimer. Riley, et al., Laboratory Medicine, Volume 47, Issue 2, 1 May 2016, Pages 90-102.

### Methods

The present disclosure provides methods for treating a biological fluid of a subject suffering from or at risk for abnormal blood clotting (e.g., PE). In some embodiments, a method for treating a biological fluid of a subject having an abnormal blood clotting comprises filtering the drawn blood of the patient using any system described herein in order to capture blood clots (e.g., clots that present a risk of pulmonary embolism) (e.g., continuously filtering over the course of a treatment).

In some embodiments, a method for treating a biological fluid of a subject having signs or symptoms of DVT (including, e.g., unexplained hypotension, unexplained tachycardia, unexplained worsening respiratory status, traditional risk factors for thrombosis (e.g., history of thrombosis, cancer, hormonal therapy), said method comprising filtering the drawn blood of the patient using any system described herein in order to capture blood clots (e.g., continuously filtering over the course of a treatment).

In some embodiments, the method comprises conducting the blood of the subject through a filter described herein that is capable of capturing blood clots. For example, the method may comprise drawing blood from a patient via a continuous-flow pump, contacting the drawn blood with filter with pore size and/or structure that retains blood clots (e.g., clots that present a risk of PE), then returning the filtered blood to the patient.

In some embodiments, the filtering step removes blood clots (e.g., thromboemboli) from the patient. In some embodiments, the method reduces clot burden in a patient. In some embodiments, the method reduces clot burden in a patient by at least 20%, at least 30%, at least 40%, at least 50%, at least 60%, at least 70%, at least 80%, at least 90%, at least 92%, at least 95%, at least 97%, or at least 99%. In some embodiments, the method removes clots from the patient before they become stuck in a blood vessel.

In some embodiments, a subject for treatment with a method and/or system of the present disclosure has or is at risk for DVT. In some embodiments, the method is performed under such conditions and for a time that all or substantial all of the thromboemboli (e.g., those above a threshold size) are removed from the DVT patient.

In some embodiments, a subject for treatment with a method and/or system of the present disclosure has COVID-19. In some embodiments, the method is performed under such conditions and for a time that the level of migrating blood clots is reduced by at least 20%, at least 30%, at least 40%, at least 50%, at least 60%, at least 70%, at least 80%, at least 90%, at least 92%, at least 95%, at least 97%, or at least 99%. In some embodiments, the method is performed under such conditions and for a time that all or substantial all of the migrating blood clots are removed from the coronavirus patient. In some embodiments, removal of migrating blood clots alleviates or improves one or more symptoms of a coronavirus patient (e.g., hypoxia, lung capacity).

In some embodiments, a subject for treatment with a method and/or system of the present disclosure has COVID-19 and is at risk for DVT. In some embodiments, a subject for treatment with a method and/or system of the present disclosure has COVID-19 and DVT. In some embodiments, the method is performed under such conditions and for a time that all or substantial all of the migrating thromboemboli are removed from the patient.

In some embodiments, provided are methods of removing abnormal clots from a subject include filtration of blood by hemodialysis-like system.

In some embodiments, a method also comprises periodically monitoring (e.g., by measuring or by obtaining a measure) D-dimer in blood of the patient. In some embodiments, periodically monitoring D-dimer is done before starting treating a patient with a system of the present disclosure and after treating the patient with said system for a period of time. In some embodiments, a measure of D-dimer is obtained using (i) quantitative fluorescence immunoassay, (ii) quantitative microparticle enzyme immunoassay (MEIA) chemiluminescence, (iii) quantitative, latex-enhanced immunoturbidimetric immunoassay, (iv) quantitative solid-phase immunochromatography, (v) enzyme-linked immunosorbent assay (ELISA), and/or (vi) quantitative latex agglutination. In some embodiments, a measure of D-dimer is obtained using a commercially available kit. Riley, et al., Laboratory Medicine, Volume 47, Issue 2, 1 May 2016, Pages 90-102.

The present disclosure encompasses a recognition that provided methods and systems are useful in patients that are ineligible for treatment with an anticoagulant or antithrombotic agent (e.g., because risks associates with anticoagulant theory may outweigh potential benefit), or in those patients where such treatment is thought to be of limited efficacy. However, in some embodiments, the present disclosure provides that anticoagulants and/or antithrombotic agents can be used in conjunction with methods of the present disclosure.

In some embodiments, the method further comprises administering an anticoagulant or antithrombotic agent to the patient. In some embodiments, an anticoagulant is or comprises one or more of: Apixaban (Eliquis), Dabigatran (Pradaxa), Edoxaban (Savaysa), Fondaparinux (Arixtra), Heparin (Fragmin, Innohep, and Lovenox), Rivaroxaban (Xarelto) and Warfarin (Coumadin, Jantoven). In some embodiments, an antithrombotic agent is or comprises aspirin, clopidogrel, and glycoprotein IIb/IIIa receptor antagonists.

### CONSTRUCTIVE EXAMPLES

The following examples are provided to illustrate, but not limit, the claimed invention.

### Example 1

The present example describes in vivo confirmation of an exemplary system of the present disclosure. Blood from swine with a procoagulation disorder (e.g., DVT) can be treated by a dialysis system of the present disclosure, which includes a continuous-flow pump and filter for capturing blood clots. D-dimer levels in the swine can be obtained before, during, and/or after treatment.

### Equivalents

It is to be understood that while the disclosure has been described in conjunction with the detailed description thereof, the foregoing description is intended to illustrate and not limit the scope of the invention(s). Other aspects, advantages, and modifications are within the scope of the following claims.

1. A system for treating a patient having or at risk of having a blood clotting disorder (e.g., wherein the patient has COVID-19), the system comprising:
a continuous-flow pump comprising an intake port and an outtake port (e.g., two ports such that it is unnecessary to create a shunt by linking an artery and a vein as with dialysis);
a disposable set comprising tubing and a filter for capturing blood clots; and
optionally, a sensor for detecting D-dimer (e.g., and/or other species) in blood as a marker of clots (e.g., a DVT marker) and/or toxic blood substances (e.g., an electrochemical impedance detector for monitoring D-dimer association with a biosensor, e.g., a single-chain antibody (ScAb) immobilized on a transducer surface with a receptor layer); and
optionally, further comprising a mechanism for signaling when the filter is to be changed.

2. The system of claim 1, further comprising an anti-thrombotic agent to reduce and/or eliminate build-up on pump surfaces that come into contact with blood (e.g., tubing interior).

3. The system of claim 2, wherein the anti-thrombotic agent comprises low-molecular weight (e.g., 6 kDa) propylene glycol alginate sodium sulfate (PSS).

4. The system of claim 1, wherein the pump is capable of providing a (e.g., maximum) continuous blood flow of at least 200 mL/min (e.g., at least 300 mL/min, at least 400 mL/min, at least 500 mL/min, at least 600 mL/min, at least 700 mL/min, at least 800 mL/min, at least 900 mL/min, at least 1000 mL/min, at least 1100 mL/min, at least 1200 mL/min, at least 1300 mL/min, at least 1400 mL/min, at least 1500 mL/min, at least 2000 mL/min, at least 2500 mL/min, at least 3000 mL/min, at least 3500 mL/min, or at least 4000 mL/min).

5. The system of claim 1, wherein the filter comprises one or more members selected from the group consisting of cellulose, synthetic polymer, and graphene (e.g., graphene for capturing very small, micrometer-scale clots) (e.g., wherein the filter has a membrane structure and/or hollow-fiber structure.

6. The system of claim 5, wherein the filter has a structure capable of capturing blood clots of size (i) 100 micrometers or larger, or (ii) 200 micrometers or larger, or (iii) 300 micrometers or larger, or (iv) 400 micrometers or larger, or (v) 500 micrometers or larger, or (vi) 600 micrometers or larger, or (vii) 800 micrometers or larger, or (viii) 1000 micrometers or larger.

7. A method of treating a patient having or at risk of having a blood clotting disorder (e.g., wherein the patient has COVID-19), the method comprising:
filtering blood of the patient using the system of claim 1 in order to capture blood clots (e.g., clots that present a risk of pulmonary embolism) (e.g., continuously filtering over the course of a treatment).

8. The method of claim 7, further comprising administering an anticoagulant to the patient.

9. The method of claim 8, wherein the anticoagulant comprises a member selected from the group consisting of heparin, warfarin (Coumadin), rivaroxaban (Xarelto), dabigatran (Pradaxa), apixaban (Eliquis), and edoxaban (Lixiana).

10. The method of claim 7, comprising monitoring for the presence of D-dimer (e.g., and/or other species) in the blood being pumped as a marker of clots and/or other toxins in the blood.

11. The method of claim 7, comprising replacing the filter (e.g., replacing the disposable set comprising the filter) upon capture of one or more clots from the blood.
